(19) United States
(12) Patent Application Publication
(10) Pub. No.: US 2025/0195616 A1
(43) Pub. Date: Jun. 19, 2025

(54) METHOD OF USING PEGYLATED INTERFERON-ALPHA

(71) Applicant: PharmaEssentia Corporation, Taipei (TW)

(72) Inventor: Ko-Chung Lin, Lexington, MA (US)

(73) Assignee: PharmaEssentia Corporation, Taipei (TW)

(21) Appl. No.: 19/059,904

(22) Filed: Feb. 21, 2025

Related U.S. Application Data
(63) Continuation of application No. 17/897,906, filed on Aug. 29, 2022.
(60) Provisional application No. 63/238,175, filed on Aug. 29, 2021.

(51) Int. Cl. A61K 38/21; A61K 47/10; A61P 37/02

(52) U.S. Cl. CPC A61K 38/212; A61K 47/10; A61P 37/02

(57) ABSTRACT

Disclosed in a method of treating a myeloid neoplasm, acute leukemia, or infectious disease in a subject, the method including administering to a subject in need thereof a pegylated interferon-α at a regular interval of every 2 to 8 weeks at a first dose of 250 to 500 μg.

### RELATED APPLICATION

This application is a continuation of U.S. patent application Ser. No. 17/897,906, filed on Aug. 29, 2022 which claims priority to U.S. Provisional Application No. 63/238,175, filed on Aug. 29, 2021, the entire content of which is hereby incorporated by reference herein.

### BACKGROUND

Classical Philadelphia chromosome-negative [Ph (−)] myeloproliferative neoplasms (MPNs) comprise three major clinical entities-polycythemia vera (PV), essential thrombocythemia (ET), and primary myelofibrosis (PMF), with the latter including pre-fibrotic PMF (prePMF). See Arber et al., Blood 2016 May 19;127(20):2391e405. These diseases are characterized by constitutive activation of Janus Kinase and Signal Transducer and Activator of Transcription (JAK-STAT) signaling pathway that is driven by mutually exclusive mutations in JAK2, MPL, and CALR, among others. See, Nangalia et al., Hematol Am Soc Hematol Edu Program 2014 Dec. 5;2014(1):287e96. Main clinical manifestations of MPN include major thrombosis, bleeding, debilitating symptoms, painful splenomegaly, and risk of secondary MF or leukemia transformation.

Consensus guideline has outlined a risk-adapted approach for the treatment of patients

with MPN. Barbui et al., Leukemia 2018 May; 32(5):1057e69. Specifically, cytoreduction is strongly recommended for high-risk ET and PV patients to avert catastrophic thrombotic events. For years, hydroxyurea (HU) and anagrelide have been most frequently used for this purpose, but these agents are not without caveats. The effect of anagrelide is limited to the control of thrombocytosis, whereas resistance or intolerance are common in patients taking HU, which also portend a poor prognosis. Although JAK inhibitor ruxolitinib has been shown to be effective as a second-line therapy for the treatment of PV, it fails to demonstrate superiority over best supportive care in patients with HU-resistant or-intolerant ET. Therefore, safer and more effective therapies are gravely needed.

### SUMMARY

In one aspect, described herein is a method of treating a myeloid neoplasm, acute leukemia, or infectious disease in a subject, the method comprising administering to a subject in need thereof a pegylated interferon-α at a regular interval of every 2 to 8 weeks for a treatment period, wherein the subject is administered a first dose of the pegylated interferon-α that is 250 to 500 μg, and wherein, prior to the first dose, the subject is interferon-treatment naive or has been administered a different pegylated interferon, the pegylated interferon-a being a conjugate of formula I:

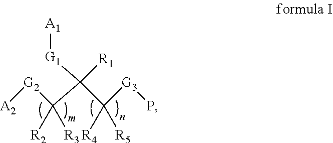

in which

each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;

each of A₁ and A₂, independently, is a polymer moiety;

each of G₁, G₂, and G₃, independently, is a bond or a linking functional group;

P is an interferon-α moiety;

m is 0 or an integer of 1-10; and

n is an integer of 1-10.

In some embodiments, the first dose is 350 to 500 μg. In some embodiments, the subject is administered a second dose of the pegylated interferon-α at 2 to 8 weeks after the first dose without an intervening dose, the second dose being 50 to 250 μg higher than the first dose and the maximum dose administered to the subject during the treatment period being no greater than 500 μg. In some embodiments, the first dose is 350 μg and the second dose is 500 μg. In some embodiments, the subject is administered a third dose of the pegylated interferon-α at 2 to 8 weeks after the second dose without an intervening dose, the third dose being 50 to 200 μg higher than the second dose. In some embodiments, the first dose is 250 μg, the second dose is 350 μg, and the third dose is 500 μg. In some embodiments, the first dose is 400 to 500 μg, which is maintained during the treatment period.

In some embodiments, the subject is resistant or intolerant to hydroxyurea or anagrelide.

In some embodiments, the conjugate has one or more properties including:

(i) a median Tmax in the range of 3 to 6 days following administration of multiple 50 to 540 μg doses of the conjugate once every two weeks to subjects;

(ii) a mean T_{1/2} in the range of 6 to 10 days following administration of multiple 50 to 540 μg doses of the conjugate once every two weeks to subjects; and

(iii) an individual maximum tolerated dose of at least 500 μg once every 2 to 4 weeks in subjects.

In some embodiments, the conjugate has one or more features including: G3 is a bond and P is an interferon-α moiety in which the amino group at the N-terminus is attached to G3; A₁ and A₂ are polyalkylene oxide moieties each having a molecular weight of 10-30 kD; each of G₁ and G₂ is

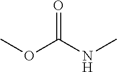

in which O is attached to A₁ or A₂, and NH is attached to a carbon atom as shown in formula I; each of R₁, R₂, R₃, R₄, and R₅ is H; m is 4 and n is 2; and the interferon-α moiety is a modified interferon-α moiety containing 1-4 additional amino acid residues. In some embodiments, the interferon-α moiety is a human interferon-α_{2b} having an extra proline residue at the N-terminus and is 166 amino acids in length. In some embodiments, the conjugate is

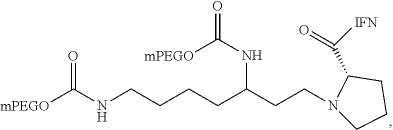

in which mPEG has a molecular weight of 20 kD and IFN is an interferon-α_{2b}.

In some embodiments, the treatment period is at least 0.5 month, at least 1 month, at least 2 months, at least 3 months, at least 6 months, at least 12 months, at least 18 months, at least 24 months, at least 30 months, at least 36 months, at least 42 months, at least 48 months, or at least 54 months or more.

In some embodiments, the subject has a myeloid neoplasm or acute leukemia, e.g., polycythemia vera, primary myelofibrosis (including pre-fibrotic primary myelofibrosis), essential thrombocythemia, or chronic myeloid leukemia.

In some embodiments, the subject has one or more responses during or by the end of the treatment period. In some embodiments, differential expression of one or more genes listed in Tables 2-6 is detected in the subject during the treatment period. In some embodiments, a decrease in one or more TNFα, TNFβ, IFNγ, IL4, and IL12 levels is detected in the subject during the treatment period. In some embodiments, an increase in hepcidin level is detected in the subject during the treatment period.

In some embodiments, the infectious disease is hepatitis B viral infection, hepatitis C viral infection, or hepatitis D viral infection. In some embodiments, the first dose is 400 to 500 μg, which is maintained during the treatment period. In some embodiments, the subject has hepatitis C viral infection and, optionally, is co-administered with Ribavirin. In some embodiments, the subject has one or more of the following responses during or by the end of the treatment period: (i) undetectable HCV RNA in serum; (ii) HBV DNA<2000 IU/mL in serum; (iii) undetectable HBV DNA in serum; (iv) hepatitis B virus surface antigen (HBsAg)<1500 IU/mL in serum; (v) normalization of alanine aminotransferase (ALT) level; and (vi) e seroconversion in hepatitis B e antigen positive (HBeAg+subject).

The details of one or more embodiments are set forth in the accompanying drawing and the description below. Other features, objects, and advantages of the embodiments will be apparent from the description and drawing, and from the claims.

### BRIEF DESCRIPTION OF DRAWINGS

FIG. 1 is a set of graphs showing hematological response in Ropeg-treated patients. The figure illustrates the fluctuation of Hct levels (a) in three patients treated for poorly controlled erythrocytosis and platelet counts (b) in five patients treated for exaggerated thrombocytosis. The horizontal axis represents the time (in weeks) following the initiation of Ropeg therapy. FIG. 2 is a set of graphs showing molecular response in eight JAK2-mutated patients. (a) Relative change in percentages of JAK2 mutant allele burden (AB) as compared with pre-treatment baseline values. Upward bars represent increase in AB, whereas downward columns indicate decreased mutant amounts. (b) The absolute values of JAK2 mutant AB over time. Data from two patients who later progressed are not included, hence only results from six cases are shown. (c) Three patterns of molecular response in six continuously treated patients. Rates of changes in mutant AB every 3 months are indicated here. Rates in the first six months of treatment are shown in light grey, whereas those beyond 6 months are demonstrated in dark grey.

FIG. 3 is a set of graphs showing assessment of clinical symptoms and spleen size before and after treatment. (a) Impacts of Ropeg treatment on symptom burdens assessed by MPN SAF scores. The dark and grey horizontal bars represent the pre-and post-treatment scores, respectively. MPN SAF, MPN symptom assessment form. (b) The changes in spleen size. The dark and grey horizontal bars indicate the pre-and post-treatment spleen indices, respectively. The spleen indices were calculated by the length of the long axis (in centimeters) multiplied by that of the short axis, with both axes crossing each other at a right angle over the splenic hilum.

FIG. 4 is a set of graphs showing effects of Ropeg on cytokine profiles. (a) Relative change in the percentages of levels of five cytokines over time in one particular patient. The plasma cytokine levels were measured with multiplex ELISA-based Q-plex™ Human Cyokine HS Screen Array (Quansys Biosciences), which contained more than a dozen of cytokines. Only cytokines with significantly altered levels after treatment are shown here. (b) The absolute values of plasma hepcidin levels over time in six PV patients. Human Hepcidin ELISA Kit (Cusabio Technology) was used for the quantification.

FIG. 5 is a graph showing distinct transcriptomic profiling before and after Ropeg therapy. The pre-and post-treatment transcript levels of three platelet-relevant genes (PPBP, PF4, and ITGA2B/CD41) and one erythroid-associated gene (TFRC/CD71) in the triple-negative ET patient are shown in the graph.

FIG. 6 is a set of graphs showing (a) changes in white blood cell count (WBC) and (b) changes in platelet count during treatment with Ropeg in three PV patients treated for poorly controlled erythrocytosis.

FIG. 7 is a set of graphs showing (a) changes in white blood cell count (WBC) and (b) changes in hemoglobin level during treatment with Ropeg in three PV patients, one ET patient, and one prePMF patient treated for poorly controlled thrombocytosis. *Heavy dash line: data in a hydroxyurea-resistant PV patient with a baseline hemoglobin level of 9-10 g/dL prior to Ropeg therapy in spite of having discontinued HU for more than two weeks. Her Hb level remained stationary throughout the course of Ropeg treatment. ^{♯}Light dash line: data in a patient with prePMF whose disease evolved to secondary MF at week 38. It was believed that his progressive disease contributed significantly to anemia.

### DETAILED DESCRIPTION

Described herein is a method of treating various diseases using a pegylated interferon-α. More specifically, the pegylated interferon-a is administered at an initial dose of at least 250 μg and then titrated to reach a target dose within weeks or a few successive doses.

A pegylated interferon-α used in any of the methods described herein can be a conjugate of formula I:

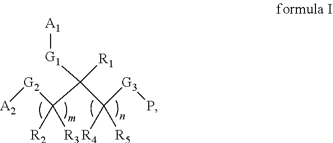

wherein each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl; each of A₁ and A₂, independently, is a polymer moiety; each of G₁, G₂, and G₃, independently, is a bond or a linking functional group; P is an interferon-α moiety; m is 0 or an integer of 1-10; and n is an integer of 1-10.

Referring to the above formula, the conjugate may have one or more of the following features: G3 is a bond and P is interferon-α moiety (e.g., a human interferon-α_{2b}) in which the amino group at the N-terminus is attached to G3; A₁ and A₂ are polyalkylene oxide moieties having a molecular weight of 2-100 kD (preferably 10-30 kD), each of G₁ and G₂ is

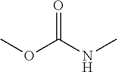

(in which O is attached to A₁ or A₂, and NH is attached to a carbon atom as shown in formula I), or each of G₁ and G₂ is urea, sulfonamide, or amide, (in which N is attached to a carbon atom as shown in formula I); m is 4, n is 2, and each of R₁, R₂, R₃, R₄, and R₅ is H; and the interferon-α moiety is a modified interferon-a moiety containing 1-4 additional amino acid residues. In some embodiments, the interferon-α moiety is a human interferon alpha-2b having an extra proline residue at the N-terminus and is 166 amino acids in length.

The conjugate may also have one or more of the following properties: (i) a median Tmax in the range of 3 to 6 days following administration of multiple 50 to 540 μg doses of the conjugate once every two weeks to subjects; (ii) a mean T_{1/2} in the range of 6 to 10 days following administration of multiple 50 to 540 μg doses of the conjugate once every two weeks to subjects; and (iii) an individual maximum tolerated dose of at least 500 μg once every 2 to 4 weeks in subjects.

In some embodiments, the conjugate is ropeginterferon alfa-2b (P1101), which has a predominant isoform having the formula:

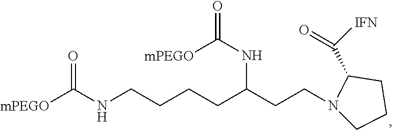

in which mPEG has a molecular weight of 20 kD and IFN is an interferon-α_{2b} (e.g., a human interferon-α_{2b}).

Ropeginterferon alfa-2b is produced by covalent attachment of a 40 kDa PEG molecule to the N-terminal proline residue of a Proline-Interferon alfa-2b (Pro-IFN alfa-2b). Proline-interferon alfa-2b is generated by recombinant DNA technology introducing an extra proline residue to a human interferon alpha-2b at N-terminus, giving a polypeptide of total 166 amino acids in length. Pro-IFN alfa-2b has a molecular weight of approximately 19 kDa and has the amino acid sequence identical to the theoretical sequence predicted excluding the additional N-terminal proline. It is then PEGylated with an approximately 40 kDa PEG moiety forming approximately 60 kDa PEGylated proline-interferon alfa-2b or ropeginterferon alfa-2b. The biological activity of ropeginterferon alfa-2b is determined by cytopathic effect (CPE)-based antiviral assay.

The conjugate of formula I is described in detail in WO2009/023826A1. In particular, WO2009/023826A1 teaches a method of making P1101.

In any of the methods described herein, the pegylated interferon-α can be administered by any means known in the art, e.g., via subcutaneous or intravenous route. The pegylated interferon-α can be formulated as an injectable formulation. For example, it can be in the form of a ready-to-use prefilled syringe (PFS) containing, e.g., 0.2 to 2 mL of solution, that can be for self-injection. Each PFS can contain the labeled amount of the drug product, sodium chloride, sodium acetate anhydrous, acetic acid, benzyl alcohol, and polysorbate 80. The vehicle for the drug product can be sterile water for injection and the drug product solution can have a pH of about 6.0.

The term “dose” refers to the amount of a compound administered to a subject at one time.

The term “interval” refers to the time between administration of two consecutive doses. In any of the methods described herein, the pegylated interferon-α is administered at an interval of 2 to 8 weeks, e.g., 2, 3, 4, 5, 6, 7, or 8 weeks. For example, a dose can be administered once every 2, 3, 4, 5, 6, 7, or 8 weeks. An interval that is defined in days or months is also contemplated. A regular interval of 10 to 60 days (e.g., 14, 21, 25, 26, 27, 28, 29, 30, 31, 35, 42, 49, and 56 days), one month, or two months can be utilized in the method.

A treatment period can be at least 1, 2, 3, 4, 5, 6, 7, 8, 9, 10, 11, 12, 24, 36, 42, 48, 54, 60, 66, 72, 78, 84 or more months. In some embodiments, the treatment period is 1, 2, 3, 4, 4.5, 5, 5.5, 6, 6.5, 7, 7.5, 8, 8.5, 9, 9.5, 10 or more years. In some embodiments, the treatment period is at least 4, 8, 12, 16, 20, 24, 28, 32, 36, 40, 44, 48, 52, 56, or 60 weeks.

A dose of the pegylated interferon-a administered during the treatment period ranges from 250 to 650 μg. The dose can be 250 μg, specifically up to 255 μg, specifically up to 260 μg, specifically up to 265 μg, specifically up to 270 μg, specifically up to 275 μg specifically up to 280 μg, specifically up to 285 μg, specifically up to 290 μg, specifically up to 295 μg, specifically up to 300 μg, specifically up to 305 μg, specifically up to 310 μg, specifically up to 315 μg, specifically up to 320 μg, specifically up to 325 μg, specifically up to 330 μg, specifically up to 335 μg, specifically up to 340 μg, specifically up to 345 μg, specifically up to 350 μg, specifically up to 400 μg, specifically up to 450 μg, specifically up to 500 μg, specifically up to 540 μg, or specifically up to 650 μg. In some embodiments, an initial (starting) dose of 250 to 500 μg (e.g., 250 μg, 300 μg, 350, 400 μg, 450 μg, or 500 μg) of the pegylated interferon-α is administered to the subject. The initial dose can be maintained or varied during the treatment period.

In any of the methods or treatment periods described herein, the pegylated interferon-α can be titrated. A subject can be treated with a lower starting dose (e.g., 250 to 500 μg) of the pegylated interferon-α. If the subject responds well (e.g., lack of significant drug-related adverse events, significant self-reported discomfort, abnormal hematological responses, or other symptoms) after a time (e.g., 2 to 8 weeks), the dose given to the subject may be increased incrementally (e.g., by 50 to 250 μg, 50 μg, 75 μg, 100 μg, 125 μg, 150 μg, 200 μg, 250 μg or a combination thereof) every 2 to 16 weeks (e.g., every 2, 3, 4, 5, 6, 7, 8, 9, 10, 11, 12, 13, 14, 15, or 16 weeks, or a combination thereof) until the dose reaches a target dose (e.g., at least 400, 425, 450, 475, 500, 525, 550, or 650 μg). After that, the target dose is maintained during the treatment period. The dose can be increased successively until the desired dose is reached. For example, if the pegylated interferon-α is administered once every 2, 3, 4, 5, 6, 7, or 8 weeks, the dose can be increased every 2, 3, 4, 5, 6, 7 or 8 weeks, respectively. In some embodiments, a subject can be given a starting dose of 250 μg (i.e., week 0). If the subject responds well to the initial dose, the dose can be increased by 100 to 150 μg every 2 to 8 weeks until it reaches a target dose of 500 μg. For example, a 250-350-500 dosing schedule can be implemented (i.e., 250 μg at week 0, 350 μg at week 2 to 8, and 500 μg at the third administration 2 to 8 weeks after the second dose, without other intervening doses). Alternative, a subject may be given a starting dose of 350 μg and a second dose of 500 μg 2 to 8 weeks thereafter without an intervening dose (i.e., 350-500). Exemplary dosing schedules can include: 250-350-500, 250-400-500, 250-400, 250-500, 250-400-500, 250-450, 250-350-400-500, 250-300-400-500, 250-350-450-500, 250-350-450, 250-250-350-500, 250-250-250-350-500, 250-350-350-500, 350-500, 350-400-500, 350-400-450-500, 350-450-500, 350-400, 350-450, 350-350-500, 350-350-350-500, 400-450-500, 400-500, 400-400-500, and 450-500. In some embodiments, the target dose is reached in 4 to 8 weeks from the initial administration of the pegylated interferon-a. During the titration process, any dose, prior to reaching the target dose, may be maintained for a time period (e.g., 4 to 16 weeks) or a number of successive doses (e.g., 2 to 8 successive doses, or 250-350-350-500) or reduced depending on the subject's response. In some embodiments, the target dose is reached within 2 to 4 successive doses.

An initial dose or starting dose of the pegylated interferon-α refers to the first dose administered to a subject during a treatment period (i.e., week 0), wherein, prior to the treatment period, the subject is interferon-treatment naïve or has not been administered the same pegylated interferon-α. A subject who is interferon-treatment naïve is a subject who has not been treated with any form of interferon, whether pegylated or non-pegylated (e.g., recombinant interferon, or peginterferon alfa-2b or peginterferon alfa-2a approved to be administered weekly). The subject treated with the pegylated interferon-α can be resistant or intolerant to hydroxyurea or anagrelide.

Myeloid neoplasms and acute leukemia can include chronic myelogenous leukemia, BCR-ABL1-positive, chronic neutrophilic leukemia, polycythemia vera, primary myelofibrosis (including pre-fibrotic primary myelofibrosis), essential thrombocythemia, chronic eosinophilic leukemia not otherwise specified, mastocytosis, myeloproliferative neoplasms unclassifiable; myeloid and lymphoid neoplasms associated with eosinophilia and abnormalities of PDGFRA, PDGFRB or FGFR1, specifically myeloid and lymphoid neoplasms associated with PDGFRA rearrangement, myeloid neoplasms associated with PDGFRB rearrangement, myeloid and lymphoid neoplasms associated with FGFR1 abnormalities; myelodysplastic/myeloproliferative neoplasms (MDS/MPN), specifically chronic myelomonocytic leukemia, atypical chronic myeloid leukemia, BCR-ABL1-negative, juvenile myelomonocytic leukemia, provisional entity: refractory anemia with ring sideroblasts and thrombocytosis; myelodysplastic syndrome (MDS), specifically refractory cytopenia with unilineage dysplasia, refractory anemia, refractory neutropenia, refractory thrombocytopenia, refractory anemia with ring sideroblasts, refractory cytopenia with multilineage dysplasia, refractory anemia with excess blasts, myelodysplastic syndrome with isolated del (5q), myelodysplastic syndrome, unclassifiable, childhood myelodysplastic syndrome; acute myeloid leukemia (AML) and related neoplasms, specifically acute myeloid leukemia with recurrent genetic abnormalities, AML with t(8;21)(q22;q22); RUNX1-RUNX1T1, AML with inv(16)(p13.1q22) or t(16;16)(p13.1;q22); CBFB-MYH11, APL with t(15;17)(q22;q12); PML-RARA, AML with t(9;11)(p22;q23); MLLT3-MLL, AML with t (6;9) (p23;q34); DEKNUP214, AML with inv (3) (q21q26.2) or t (3;3) (q21;q26.2); RPN1-EVI1, AML (megakaryoblastic) with t(1;22)(p13;q13); RBM15-MKL1, acute myeloid leukemia with myelodysplasia-related changes, therapy-related myeloid neoplasms, acute myeloid leukemia, not otherwise specified, AML with minimal differentiation, AML without maturation, AML with maturation, acute myelomonocytic leukemia, acute monoblastic/monocytic leukemia, acute erythroid leukemia, pure erythroid leukemia, erythroleukemia, erythroid/myeloid, acute megakaryoblastic leukemia, acute basophilic leukemia, acute panmyelosis with myelofibrosis, myeloid sarcoma, myeloid proliferations related to Down syndrome, transient abnormal myelopoiesis, myeloid leukemia associated with Down syndrome, blastic plasmacytoid dendritic cell neoplasm; acute leukemias of ambiguous lineage, specifically acute undifferentiated leukemia, mixed phenotype acute leukemia with t(9;22)(q34;q11.2); BCR-ABL1, mixed phenotype acute leukemia with t(v;11q23); MLL rearranged, mixed phenotype acute leukemia, B-myeloid, NOS, mixed phenotype acute leukemia, T-myeloid, NOS, B lymphoblastic leukemia/lymphoma, specifically B lymphoblastic leukemia/lymphoma, NOS, B lymphoblastic leukemia/lymphoma with recurrent genetic abnormalities, B lymphoblastic leukemia/lymphoma with t(9;22)(q34;q11.2);BCR-ABL 1, B lymphoblastic leukemia/lymphoma with t(v;11q23);MLL rearranged, B lymphoblastic leukemia/lymphoma with t(12;21)(p13;q22) TEL- AML1 (ETV6-RUNX1), B lymphoblastic leukemia/lymphoma with hyperdiploidy, B lymphoblastic leukemia/lymphoma with hypodiploidy, B lymphoblastic leukemia/lymphoma with t (5;14) (q31;q32) IL3-IGH, B lymphoblastic leukemia/lymphoma with t (1;19) (q23;p13.3);TCF3-PBX1.

Response criteria for assessing treatment can include symptoms and signs of the disease, peripheral blood counts (e.g., platelet counts and white blood cell counts), vascular events, signs of progression of disease, bone marrow histology, molecular response, and cytogenic response. Response criteria can be defined based on consensus criteria in the art, e.g., the European LeukemiaNet (ELN) and/or International Working Group (IWG) criteria.

For example, any combination of the following criteria can be used to define a response for essential thrombocythemia or polycythemia vera: resolution of disease-related signs including palpable hepatosplenomegaly; large symptoms improvement; platelet count ≤400×10⁹/L; white blood cell count <10×10⁹/L; hematocrit <45% (with or without phlebotomy in the previous 3 months or 12 weeks); absence of leukoerythroblastosis; absence of signs of progressive disease; absence of any hemorrhagic or thrombotic events; bone marrow histological remission (e.g., disappearance of megakaryocyte hyperplasia and absence of >grade 1 reticulin fibrosis; or presence of age-adjusted normocellularity and disappearance of trilinear hyperplasia, and absence of >grade 1 reticulin fibrosis); and molecular remission or response. A complete response (e.g., a complete hematological response) can be defined to include all or a subset of the criteria. A partial response (e.g., a partial hematological response) can be defined to include a smaller subset of the criteria.

For myelofibrosis (MF) (e.g., associated with primary MF, post-polycythemia vera MF, and post-essential thrombocythemia MF), any combination of the following criteria can be used to define a response: age-adjusted normocellularity; <5% blasts; ≤grade 1 MF; hemoglobin ≥100 g/L and <UNL; neutrophil count ≥1×109/L and <UNL; platelet count ≥100×10⁹/L and <UNL; <2% immature myeloid cells; resolution of disease symptoms; spleen and liver not palpable; no evidence of extramedullary hematopoiesis (EMH); hemoglobin ≥85 but <100 g/L and <UNL; platelet count ≥50, but <100×10⁹/L and <UNL; achievement of anemia, spleen or symptoms response without progressive disease or increase in severity of anemia, thrombocytopenia, or neutropenia; transfusion-independent patients: a >20 g/L increase in hemoglobin level; transfusion-dependent patients: becoming transfusion-independent; a baseline splenomegaly palpable at 5-10 cm, below the LCM, becomes not palpable; a baseline splenomegaly palpable at 22 10 cm, below the LCM, decreases by ≥50%; ≥35% spleen volume reduction; ≥50% reduction in the MPN-SAF TSS; cytogenic remission or response; and molecular remission or response.

For chronic myeloid leukemia, any combination of the following criteria can be used to define a response: platelet count ≤400×10⁹/L; white blood cell count ≤10×10⁹cells/L; less than 5% basophils in peripheral blood; absence of extramedullary involvement; absence of immature granulocytes (such as blasts, promyelocytes, and myelocytes); absence of splenomegly; molecular remission or response; and cytogenic remission or response.

A molecular response can include a reduction of a mutant allele burden. For example, a molecular response can include a reduction in one or more of JAK2617F allele burden, CALR mutant allele burden, and MPL mutant allele burden. A reduction in non-driver mutant allele burdens (e.g., TET2 mutant allele burden) may also be an indication of a molecular response.

Allele burden (%) over time can be calculated. Allelic burden represents the percentage of mutant alleles present among all alleles of a particular gene in peripheral blood mononuclear cells. More specifically, the reduction of the allele burden can be at least 20%, 25%, 30%, 35%, 40%, 45%, 50%, 55%, 60%, 65%, 70%, 75%, 80%, 85%, 90%, 95%, or 99% or more, specifically between two time points or within a treatment period. The allele burden can decline to 50% or less, e.g., less than 50%, 45% or less, 40% or less, 37% or less, 35% or less, 30% or less, 25% or less, 20% or less, 15% or less, 10% or less, 7% or less, 5% or less, or 1% or less. A complete molecular response (CMR) is achieved when the allele burden is below the threshold of 1%.

In the case of chronic myeloid leukemia, a molecular response includes a reduction of BCR-ABL1 transcripts to a particular level according to the international scale (IS). For example, a molecular response can be a reduction of BCR-ABLI transcripts to ≤0.1% or deeper, ≤0.01% or deeper, ≤0.0032% or deeper, ≤0.001% or deeper, or a non-detectable level. A cytogenic response is determined by evaluation of percentages of cells containing the Philadelphia (Ph) chromosome in bone marrow samples. At least 20 dividing cells (metaphases) should be analyzed. The presence of greater than 95% Ph+ cells can be considered as a non-response. A partial cytogenic response can be 1% to 35% Ph+ cells. A complete cytogenic response (CCyR) is defined as 0% Ph+ cells.

Other indications of a good response can include a normal spleen size (measured via ultrasound; ≤12 cm for females, ≤13 cm for males), absence or low rate of any thromboembolic events, and a reduction of phlebotomy requirements by at least 50%, e.g., at least 55%, 60%, 65%, 70%, 75%, 80%, 85%. 90%, 95%, or 99%. A subject can be free of phlebotomy. Myeloproliferative Neoplasm Symptom Assessment Form Total Symptom Score (MPN-SAF TSS) can also be used to assess a subject's response (i.e., symptom improvement). A reduction in TSS score (e.g., by at least 2 points, at least 5 points, at least 10 points, or at least 15 points, or reduction to a score of ≤10) can indicate an improvement.

A subject may also have one or more of the following responses during or by the end of the treatment period: (i) differential expression of one or more genes listed in Tables 2-6; (ii) a decrease in one or more cytokine levels (e.g., TNFα, TNFβ, IFNγ, IL4, and IL12); and (iii) an increase in hepcidin level.

The change in a response can be determined by comparing responses at two time points, one of which can be before the initiation of the treatment. The change can be statistically significant or to any extent (e.g., by 5 to 100%, or by 1 to 20 folds).

Infectious diseases include hepatitis B infection, hepatitis C infection and hepatitis D infection (e.g., chronic hepatitis B, chronic hepatitis C, or chronic hepatitis D). A subject being treated can have one or more of the following responses during or by the end of the treatment period: (i) undetectable HCV RNA in serum; (ii) HBV DNA <2000 IU/mL in serum; (iii) undetectable HBV DNA in serum; (iv) hepatitis B virus surface antigen (HBsAg) <1500 IU/mL in serum; (v) normalization of alanine aminotransferase (ALT) level; and (vi) e seroconversion in hepatitis B e antigen positive (HBeAg+ subject).

Any of the above responses (e.g., hematological response, molecular response, and gene expression) can occur or be detected in a subject by week 4, 8, 12, 16, 20, 24, 28, 32, 36, 40, 44, or 48, by month 3, 6, 12, 18, 24, 30, 36, 42, or 48, or by year 1, 2, 3, 4, 5, or 6 after initiation of the treatment. Any of the responses can be maintained or further improved thereafter throughout the treatment period or beyond.

A subject treated with a conjugate of formula I can exhibit less frequent adverse events (e.g., 5% to 100%, 10% to 30%, 20% to 40%, 30% to 50%, 40% to 60%, or 50% to 70% less of total adverse events, any adverse events, ≥grade 3 events, or ≥grade 4 events) or lower grade events (e.g., absence of ≥grade 3 events) than a subject treated with a different pegylated interferon.

Adverse events can include hematologic, non-hematologic, or biochemical adverse events. Hematologic adverse events can include anemia, neutropenia, lymphopenia, thrombocytopenia, and pancytopenia. Non-hematologic adverse events can include infections, psychiatric disorders (e.g., depression), asthenia, fatigue, musculoskeletal pain, muscle cramps, abdominal pain, edema, dizziness, rash, headache, nausea, thrombosis, weight gain, weight loss, seizures, hemorrhage, diarrhea, and vomiting. Biochemical events can include elevated aspartate aminotransferase, alanine aminotransferase, and gamma-glutamyltransferase levels. Adverse events are graded based on standards accepted in the field (e.g., National Cancer Institute Common Terminology Criteria for Adverse Events).

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications cited herein are herein incorporated by reference in their entirety.

### Example 1: Materials and methods

### Patients

Patients with Ph (−) myeloproliferative neoplasms (MPN) from Chang-Gung Memorial Hospital, Chiayi, Taiwan, who were enrolled in a compassionate use program (CUP) provided by the manufacturer (PharmaEssentia; Taipei, Taiwan) of ropeginterferon alfa-2b (Ropeg) were included in this report. To be eligible, patients must be resistant or intolerant to currently available therapies for MPN in Taiwan—mainly HU and anagrelide. They could be ruxolitinib-naïve, since this agent has not been reimbursed in Taiwan. Patients with autoimmune disorders, psychiatric illness, and acute or chronic infections were carefully screened and excluded from the treatment. Each case was independently reviewed and approved for use of this agent by both the Institutional Review Board (IRB) and the Ministry of Health and Warfare in Taiwan.

Driver mutations and dosing schedule

Relevant clinical data before and after treatment were collected. Detection of the driver mutations in these patients, namely JAK2V617F, CALR, and MPL, was performed as described previously. See Chen et al., Haematologica 2017 March;102(3):509e18; and Hsu et al., Haematologica 2018 October;103 (10):e450e4. Moreover, MPN symptom assessment form total symptom score (MPN-SAF TSS) was used to evaluate the symptomatic burden of each patient. Ropeg was given every 2 weeks at a starting dose of 250 μg. If there were no significant drug-related adverse events, either self-reported discomforts by the patients or abnormalities in biochemical or hematological profiles, the dose of this agent would be increased by 100 μg every two weeks until it reached the target dose of 500 μg on week 6.

Treatment efficacy and toxicity

For response evaluation, the MPN-SAF TSS was recorded periodically. Spleen size was assessed by abdominal echography, and spleen indices were calculated by the length of the long axis (in centimeter) multiplied by that of the short axis, with both axes crossing each other at a right angle over the splenic hilum. A spleen index between 20 and 24 suggested borderline splenomegaly, whereas an index above 24 was considered to represent splenomegaly with clinical significance. JAK2V617F mutant allele burdens were quantified every 3 months. See Hsu et al., Haematologica 2018;103:e450-e4. Moreover, hemograms, biochemistry profiles, and adverse events (AEs) were routinely monitored and collected at every visit for each patient. The rate of peripheral blood count complete remission (PBCR) was also evaluated, which assessed the possibility of normalized hemogram, through the employment of the 2013 ELN and IWG-MRT consensus response definition in blood indices without the spleen, bone marrow, and symptom criteria: platelet count ≤400×10⁹/L, WBC count <10×10⁹/L, and absence of leukoerythroblastosis. See Barosi et al., Blood 2013;121:4778-81. All AEs were graded according to the National Cancer Institute's Common Terminology Criteria for Adverse Events version 5.0 (CTCAE v5.0). For better assessment of efficacy, only patients who were treated with this agent for at least 3 months were included.

Cytokine array analysis and quantification of FGF2, VEGF and hepcidin

To quantify plasma cytokine levels, the multiplex ELISA-based Q-Plex™ Human Cytokine HS Screen Array (Quansys Biosciences, Logan, Utah, USA) was employed, which contained IL-1α, IL-1β, IL-2, IL-4, IL-5, IL-6, IL-10, IL-12p70, IL-13, IL-15, IL-17, IL-23, IFN-γ, TNF-α, and TNF-β. The Q-plex analysis was performed in a 96-well plate following the manufacturer's protocol. Data capture was performed by Q-View™ Imager Pro, and the Q-View™ Software was utilized for final analysis. On the other hand, the FGF2 and VEGF concentrations were measured with the PicoKine™ ELISA kit (BOSTER BIOLOGICAL TECHNOLOGY, Pleasanton, CA, USA), whereas quantification of the hepcidin levels was performed with Human hepcidin ELISA Kit (CUSABIO TECHNOLOGY, Houston, TX, USA).

RNA sequencing

Peripheral blood granulocytes were harvested from whole blood with Ficoll 400 (United States Biological, MA, USA). Total RNA was extracted using the TRI Reagent® (Sigma-Aldrich, MO, USA). Following quality control (QC), RNA samples were prepared according to the official protocol of Illumina. Agilent's SureSelect Strand-Specific RNA Library Preparation Kit (Agilent Technologies, Santa Clara, CA, USA) was used for library construction, which was followed by AMPure XP Beads (Beckman Coulter, Taipei, Taiwan) size selection and Illumina's sequencing-by-synthesis (SBS) technology. Sequencing data (FASTQ files), QC, and sequencing trimming processes were generated by Welgene's pipeline based on Illumina's base-calling program bc12fastq v2.2.0 (WELGENE Biotech, Taipei, Taiwan). HISAT2 was applied to sequence mapping alignment (HISAT2 available at ccb.jhu.edu). Analysis of differentially expressed genes with genome bias detection/correction were performed on cuffdiff and Welgene in-house programs (WELGENE Biotech). See, Trapnell et al., Nat Protoc 2012;7:562-78. Log₂(fold changes) beyond >1 or <−1 and a p-value of less than 0.01 was set as the cutoff for identifying differentially expressed genes. Gene enrichment analysis was carried out by employing clusterProfiler v.3.6 (available at bioconductor.org) for functional studies.

### Example 2: Results

Baseline characteristics of the patients

In all, nine treated patients were included in the analysis. Table 1 shows their baseline characteristics. The diagnosis was polycythemia vera (PV) in 6 patients, whereas the remaining 3 cases had essential thrombocythemia (ET), pre-fibrotic primary myelofibrosis (prePMF), and post-ET myelofibrosis (MF), respectively. Eight out of the nine patients had JAK2-mutated MPN, and one patient had triple-negative (TN) disease. The spleen was enlarged in eight patients (89%). The main problem that led to the use of Ropeg was poorly controlled erythrocytosis in three and thrombocytosis in five patients. The last one was a young lady who, in spite of having low-risk post-ET myelofibrosis, asked for disease-specific treatment and was granted approval upon careful review.

TABLE 1
Baseline patient characteristics

| | | | | | | | Disease |
|---|---|---|---|---|---|---|---|
| | | | | Driver | | Main | duration |
| Case | Age | Gender | Diagnosis | mutation | Splenomegaly | problem^{a} | (yrs) |
| 1 | 78 | M | PV | JAK2V617F | Yes | Hct | 1.2 |
| 2 | 70 | M | PV | JAK2V617F | Yes | Hct | 0.3 |
| 3 | 50 | M | PV | JAK2V617F | Yes | Hct | 8.4 |
| 4 | 37 | F | PV | JAK2V617F | Yes | Platelet | 2.8 |
| 5 | 61 | F | PV | JAK2V617F | Yes | Platelet | 6.7 |
| 6 | 38 | F | PV | JAK2V617F | Yes | Platelet | 4.8 |
| 7 | 36 | M | ET | TN^{b} | No | Platelet | 12.9 |
| 8 | 65 | M | PrePMF | JAK2V617F | Yes | Platelet | 5.4 |
| 9 | 30 | F | Post-ET MF | JAK2V617F | Yes | MF | 4.0 |

^{a}The main problem that led to the use of Ropeg; three were treated for poorly controlled erythrocytosis (Hct: hematocrit), and five were treated for poorly controlled thrombocytosis (platelets).
^{b}TN: triple negative (absence of JAK2V627F, CALR and MPL mutations).

The median duration of treatment with Ropeg was 54 weeks (range 26-90 weeks). At the time of the most recent follow-up, seven out of the nine patients received continuous therapy. Two patients stopped the treatment because of disease progression, one (PV) with acute myeloid leukemia (AML) transformation on week 26, and the other (prePMF) with disease evolution to MF on week 38. Significantly, unlike the patient population enrolled in the prospective PROUND/CONTI-PV trial in which many of them were treatment-naïve, most of the patients here had run out of options for their refractory disease, indicating the substantial potency of this novel agent.

Efficacy in the control of hemogram

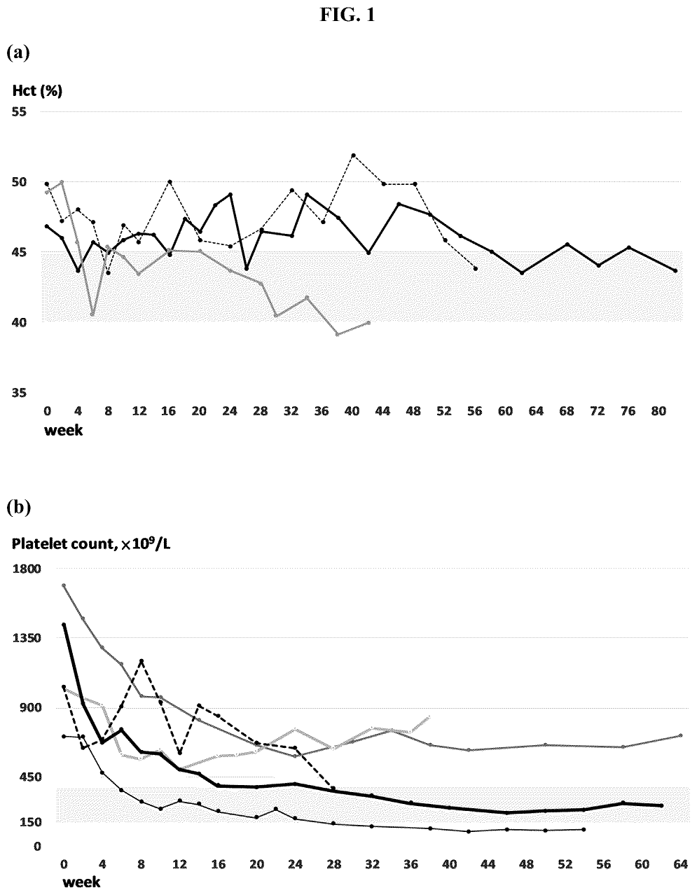

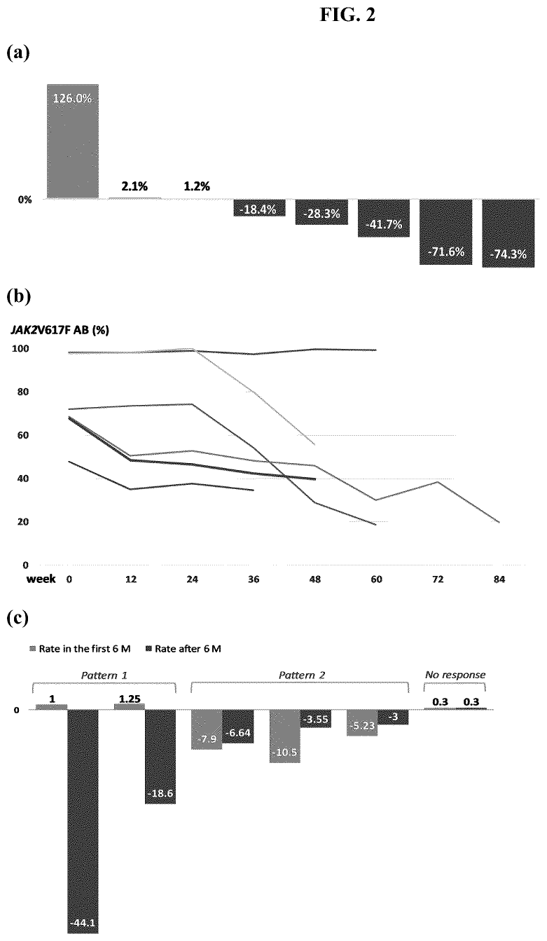

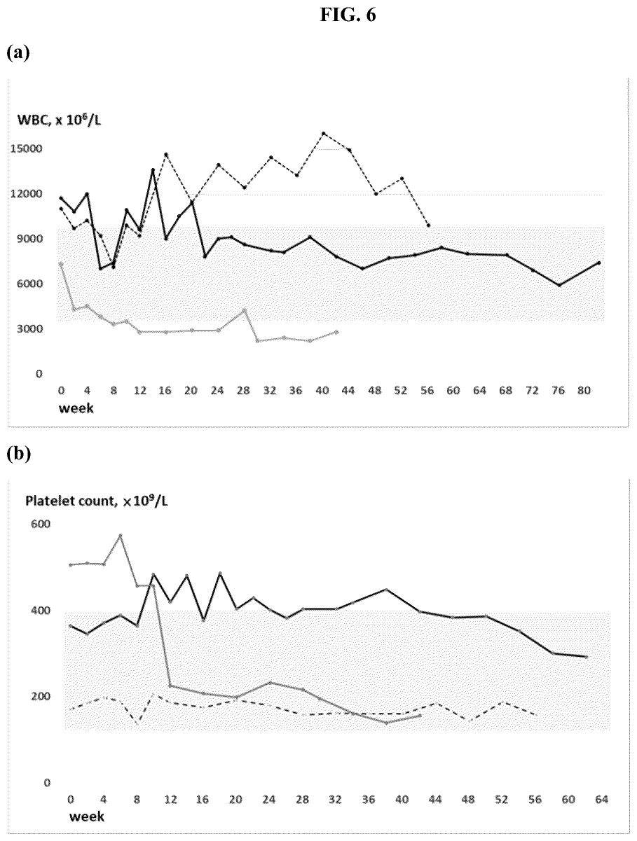

The hematocrit (Hct) levels in three PV patients treated for poorly controlled erythrocytosis are shown in FIG. 1(a). Due to preceding therapy with concurrent hydroxurea and phlebotomies, the baseline Hct levels prior to first dose of Ropeg were not exaggeratingly high. For cases 01 and 03, although the Hct control seemed unsatisfactory, this agent did reduce the frequencies of phlebotomy by 83% and 50%, respectively. After a 56-week treatment period, case 01 finally achieved peripheral blood count complete remission (PBCR) as defined by the ELN response criteria. See, Barosi et al., Blood 2013 Jun. 6;121(23):4778e81. On the other hand, the response was more robust in case 02, who received phlebotomy only once on week 2 and had his Hct level fall below 45% by week 10. WBC and platelet counts of the three patients are shown in FIG. 6.

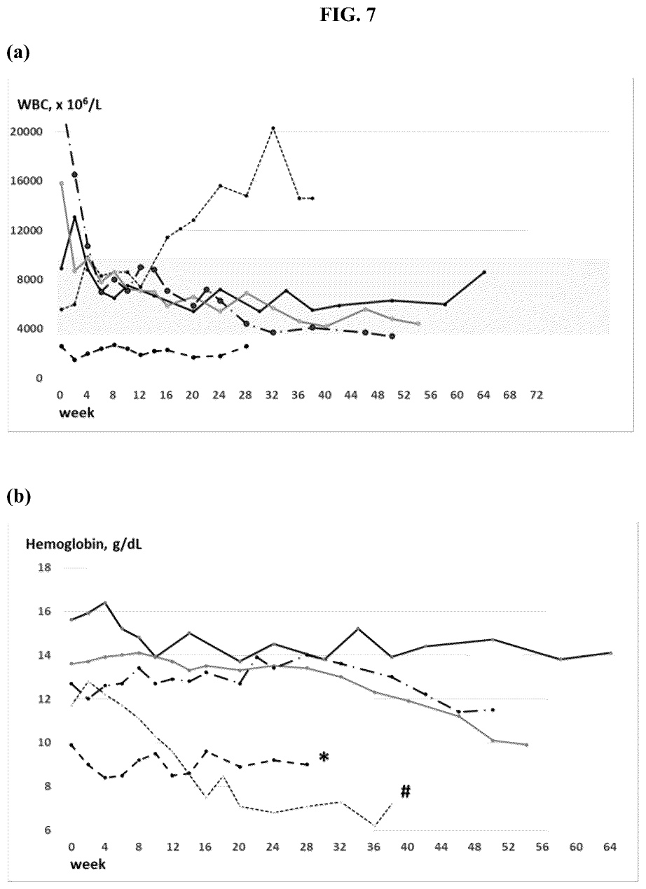

In comparison with what was observed in the control of erythrocytosis, there was rapid and dramatic decline in the platelet counts after treatment with this agent in all five patients with refractory thrombocytosis, mostly within the first 8 weeks of therapy. See FIG. 1(b). WBC and platelet counts of the five patients are shown in FIG. 7. Importantly, three patients achieved PBCR. In all three, the time to PBCR was fairly short (6, 16, and 28 weeks after treatment, respectively). The results suggest that this agent might hold great promise in the control of thrombocytosis, an area yet to be explored in a prospective clinical trial.

In all, among the eight evaluable patients, five patients (62.5%) achieved PBCR after a median of 16 weeks (range: 6-56 weeks), whereas one patient was deemed as a nonresponder in terms of hemogram control.

Changes in the clonal size of MPN patients

To assess whether Ropeg targets the mutant clones, mutant allele burden (AB) in the eight JAK2-mutated patients was quantified with a sensitive real-time PCR assay. Molecular response was documented in five (62.5%) patients. See FIG. 2(a). The percentages of reduction were estimated at 74.3%, 71.6%, 43.1%, 41.7%, and 28.3%, respectively. The changes in mutant AB over time in the six JAK2-mutated, continuously treated patients are shown in FIG. 2(b). There was steady and continuous decline in the mutant AB of the young patient with post-ET MF, implicating this agent might offer a golden opportunity in delaying disease progression in MF patients. On the other hand, JAK2 mutant AB reduction was not seen until after six months into the treatment in two patients. The rates of changes every 3 months in mutant AB showed two patterns of molecular response. See FIG. 2(c). In patients with pattern 1 response, two slow responders had stationary mutant AB in the first 6 months, which was followed by significant reduction after 6 months. On the contrary, three pattern 2 responders enjoyed steady improvement throughout the course of treatment, but the rates of mutant AB decline did not differ significantly between the first 6 months and beyond that. The findings in pattern 1 responders echoed previous experience in the phase III PROUD/CONTI-PV study, in which the superiority of Ropeg over HU in PV patients could not be demonstrated until its extended use beyond two years.

Amelioration of symptoms and reduction of spleen size

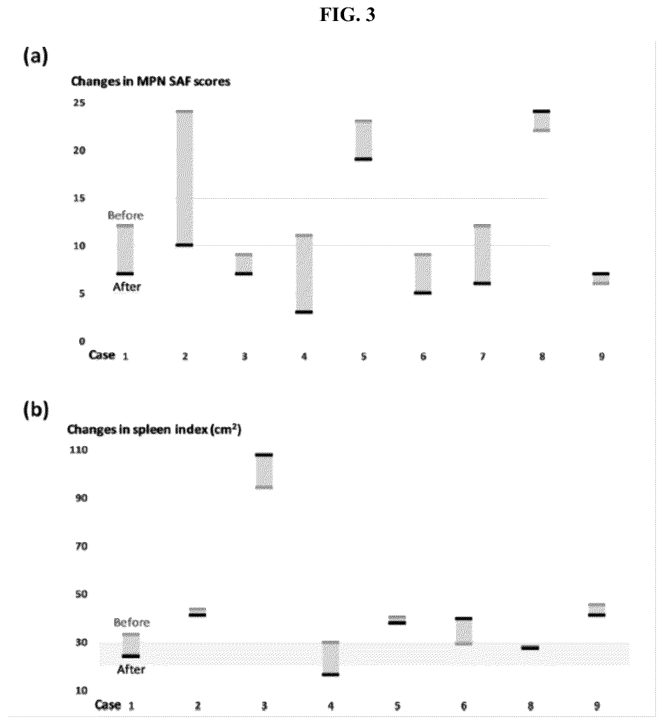

Using the MPN-SAF total symptom score, the symptomatic burden of each patient was evaluated. Not surprisingly, two patients (case 05 and case 08) with progressive disease had higher initial symptom scores and could not have their discomforts ameliorated by this agent. See FIG. 3(a). On the other hand, responders who were more symptomatic at baseline (with an initial symptom score of more than 10) were more likely to achieve significant improvement after treatment. Overall, six of the seven continuously treated patients responded well, whereas the remaining patient (case 09) did not manifest prominent disease-associated discomforts. Eight out of the nine treated patients had pre-treatment splenomegaly. Among them, two (25%) had excellent spleen response shown by nearly complete normalization of post-treatment spleen indices. See FIG. 3(b). It was probably not a coincidence that the spleen response occurred only after their JAK2 mutant AB fell below 20%, and they were the two patients with the best molecular response (74.3% and 71.6% reduction as compared with pre-treatment levels, respectively, FIG. 2A).

Effects on cytokine profiles

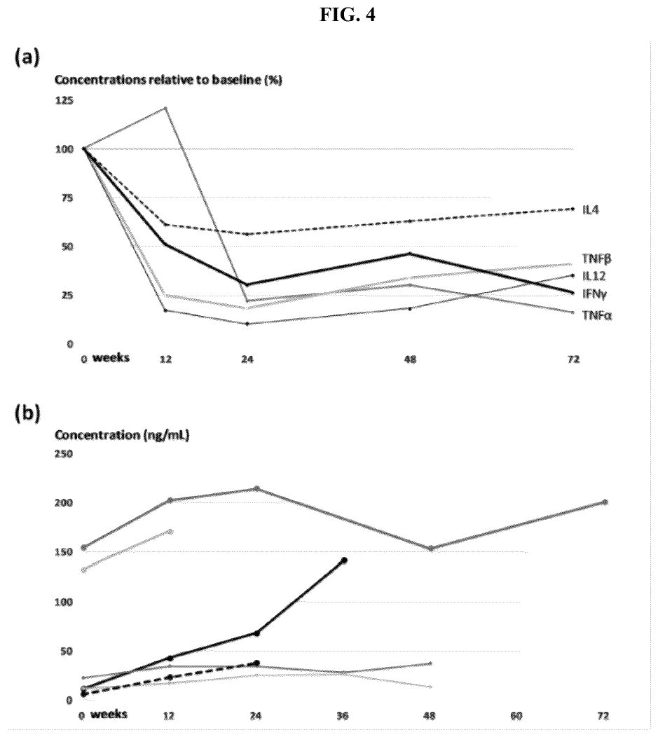

MPN patients are characterized by high plasma levels of inflammatory cytokines that contribute to debilitating constitutional symptoms. It was investigated whether Ropeg could exert some effects in subduing the cytokine storm in these patients. Gradual but significant attenuation in the plasma levels of TNFα, TNFβ, IFNγ, IL4, and IL12 was observed in case 01, who happened to enjoy drastic reduction in spleen size and JAK2 mutant AB (below 20%). See FIG. 4(a). Remarkably, he also had considerable improvement in symptom scores. On the other hand, no specific patterns of alteration in the cytokine levels were observed (data not shown) in the remainder of the patients.

Hepcidin is a master regulator of iron metabolism. See Casu et al., Blood 2018 Apr. 19;131(16):1790e4. Decreased hepcidin level has been documented in patients with JAK2-mutated PV, whereas pan-JAK inhibitor ruxolitinib has been shown to increase plasma hepcidin concentrations in patients with PV. See, Verstovsek et al., Leuk Res 2017 May;56:52e9; and Ginzburg et al., Leukemia 2018 October;32(10): 2105e16. To delineate whether Ropeg therapy alters iron metabolism, the changes in plasma hepcidin levels in the six JAK2-mutated PV patients were determined. As shown in FIG. 4(b), the hepcidin levels increased, although to various degrees, in four (66.7%) of these six patients. The data suggest that the suppression of hematopoiesis by Ropeg could have the potential to restore hepcidin-mediated regulation of erythropoiesis.

Inflammation is a well-known factor that drives up hepcidin, and MPN is characterized as a state of chronic inflammation. However, this is paradoxical to the central dogma of excessive erythropoiesis in PV, since hepcidin is the major culprit in anemia of inflammation. In fact, studies have shown that, through erythroid hyperplasia, patients with PV exhibit decreased circulating iron and increased erythroferrone levels. As a result, their hepcidin levels are suppressed. The relatively low plasma ferritin levels in PV patients suggest that inflammation which accompanies PV fails to counteract hepcidin repression by iron-restricted erythropoiesis. The aberrantly inflammation-insensitive erythropoiesis in PV may divert iron homeostasis away from other cellular functions in favor of hemoglobin synthesis, and it is plausible that reduced hepcidin expression could be part of the mechanism utilized by JAK2-mutated cells for enhanced erythropoiesis. In a proof-of-concept study, investigators have demonstrated that administration of hepcidin agonists in PV mice significantly attenuates erythrocytosis and splenomegaly, an effect considered to mediate through sequestration of iron in macrophages that prevents its utilization by erythrons for hemoglobin synthesis. Therefore, the increased hepcidin levels in a majority of our Ropeg-treated PV patients could have led to normalization of iron metabolism and restoration of tightly regulated hematopoiesis.

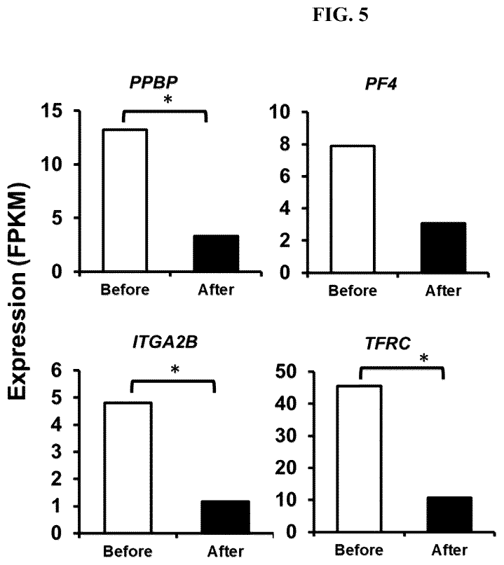

Differentially expressed genes following treatment in a triple-negative ET patient To assess the alterations in gene expression profiling following Ropeg treatment, RNA sequencing was performed in one selected patient. Due to its lack of driver mutation, the triple-negative ET sample was chosen for further investigation to better appreciate its molecular background and potential consequences after treatment. Overall, 802 genes with differential expression before and after treatment were identified with FPKM (Fragments Per Kilobase of transcript per Million mapped reads). 287 genes were statistically significant. Based on KEGG database and gene set enrichment analysis (GSEA), these genes were enriched in several biological processes, including interferon response, immune and inflammatory pathway, cell cycle, proliferation, cell division, apoptosis, and myeloid differentiation. See Tables 2-6. Furthermore, focus was put on genes that were highly enriched in the granulocytes of MPN patients, including three platelet-relevant genes (PPBP, PF4, and ITGA2B/CD41) and one erythroid-associated gene (TFRC/CD71). It was observed that these transcripts reduced significantly after treatment. See FIG. 5. The results suggest that Ropeg therapy not only induces cellular apoptosis but also balances biased differentiation within the hematopoietic hierarchy of MPN patients.

Adverse effects and dosing

Overall, the administration of Ropeg was well tolerated, as most of the treatment-related AEs were minor. See Table 7. There were no unbearable side effects that led to treatment discontinuation. Among the four grade 3/4 hematological AEs, one grade three anemia occurred in the patient with prePMF whose disease later evolved to secondary MF (on week 38), and it was reasonable to believe that his progressive disease contributed significantly to anemia. A hydroxyurea-resistant PV patient had a baseline white cell count of around 2.5×10⁹/L prior to Ropeg therapy despite having discontinued HU for more than two weeks. See FIG. 7(a), heavy dash line at the bottom of the figure. Although her WBC remained stationary throughout the course of Ropeg treatment, it was still recorded as a grade three leukopenic event. The remaining two grade 3-4 hematological AEs occurred in the same patient with post-ET MF, who was expected to have less adequate marrow reserve. This suggests that careful dose titration of Ropeg is warranted when administering this agent to patients with MF. On the other hand, there was only one grade 3 non-hematological toxicity (transaminitis). This occurred in a 78-year-old PV patient who also had fatty liver. Careful monitoring allowed continuous administration (although at only half of the target dose) of Ropeg in this patient without further deterioration of hepatic function.

TABLE 2
Differentially expressed genes enriched in cell cycle, proliferation and cell division

| Description (GO ID) | p-adjust | Genes |
|---|---|---|
| Somatic stem cell | 0.0008 | FGFR2, RAB10, DOCK7, FGF13, KIT |
| division | | |
| (GO: 0048103) | | |
| DNA synthesis | 0.0026 | BRCA1, RFC2, POLE2, NBN, TRIM25, UBE2L6, RMI1, |
| involved in DNA | | ISG15 |
| repair | | |
| DNA packaging | 0.0027 | HIST1H1D, HIST1H2BD, HIST1H4H, HIST1H2AC, |
| complex | | HIST1H2BC, HIST2H2BE, HIST1H2BG, HIST1H3E, |
| (GO: 0044815) | | HIST1H4E, HIST1H2BF |
| Stem cell division | 0.00842 | FGFR2, RAB10, DOCK7, FGF13, KIT |
| (GO: 0017145) | | |
| Negative regulation | 1.2E−13 | LTF, EIF2AK2, OAS1, APOBEC3H, OAS3, OAS2, TRIM25, |
| of viral life cycle | | TRIM6, EIF2AK4, BST2, TRIM21, RSAD2, OASL, |
| (GO: 1903901) | | RNASEL, IFITM3, IFIT5, MX1, IFI16, PARP10, PRKN, |
| | | ISG15, PLSCR1, APOBEC3G |
| Mitotic cell cycle | 0.0109 | CDC14B, PNPT1, NKX3-1 |
| arrest | | |
| (GO: 0071850) | | |
| Positive regulation | 0.0156 | NPM2, INSR, PLCB1 |
| of meiotic cell cycle | | |
| (GO: 0051446) | | |
| Stem cell | 0.0254 | EIF2AK2, FGFR2, RAB10, DOCK7, FGF13, CX3CR1, |
| proliferation | | WNT10B, KCTD11 |
| (GO: 0072089) | | |
| G-protein coupled | 0.0205 | CCL2, NPR3, PDE4D, OPRL1, ADM2, ANXA1, CYSLTR2, |
| receptor signaling | | GRM2, ABCA1, S1PR1, CYSLTR1, NPBWR1, CNR2, |
| pathway, coupled to | | SSTR3 |
| cyclic nucleotide | | |
| second messenger | | |
| (GO: 0007187) | | |

TABLE 3
Differentially expressed genes enriched in interferon response

| Description (GO ID) | p-adjust | Genes |
|---|---|---|
| Response to type I | 1.5E−15 | SP100, IFI35, OAS1, SAMHD1, OAS3, OAS2, STAT1, |
| interferon | | IFIT3, IFIT2, EGR1, TRIM6, ZBP1, IFI6, BST2, XAF1, |
| (GO: 0034340) | | RSAD2, OASL, RNASEL, IFITM3, MX1, IFI27, FADD, |
| | | TRIM56, STAT2, USP18, IRF7, ISG15 |
| Type I interferon | 1.5E−15 | SP100, IFI35, OAS1, SAMHD1, OAS3, OAS2, STAT1, |
| signaling pathway | | IFIT3, IFIT2, EGR1, TRIM6, ZBP1, IFI6, BST2, XAF1, |
| (GO: 0060337) | | RSAD2, OASL, RNASEL, IFITM3, MX1, IFI27, FADD, |
| | | STAT2, USP18, IRF7, ISG15 |
| Cellular response to | 1.5E−15 | SP100, IFI35, OAS1, SAMHD1, OAS3, OAS2, STAT1, |
| type I interferon | | IFIT3, IFIT2, EGR1, TRIM6, ZBP1, IFI6, BST2, XAF1, |
| (GO: 0071357) | | RSAD2, OASL, RNASEL, IFITM3, MX1, IFI27, FADD, |
| | | STAT2, USP18, IRF7, ISG15 |
| Response to | 3E−06 | SP100, OAS1, CCL2, OAS3, OAS2, STAT1, KYNU, |
| interferon-gamma | | GBP1, TRIM25, NMI, MT2A, BST2, TRIM21, OASL, |
| (GO: 0034341) | | CASP1, IFITM3, SNCA, CDC42EP2, IFNGR2, PARP14, |
| | | GBP6, IRF7, TRIM34, CCL4L1 |
| Interferon-gamma- | 3E−05 | SP100, OAS1, OAS3, OAS2, STAT1, GBP1, TRIM25, |
| mediated signaling | | NMI, MT2A, TRIM21, OASL, IFNGR2, PARP14, IRF7, |
| pathway | | TRIM34 |
| (GO: 0060333) | | |
| Response to | 7E−05 | STAT1, TRIM6, BST2, XAF1, PNPT1, IFITM3, AIM2, |
| interferon-beta | | PLSCR1 |
| (GO: 0035456) | | |
| Type I interferon | 9E−05 | TLR8, DDX58, DHX58, IFIH1, STAT1, TRIM25, NMI, |
| production | | ZBP1, TRIM21, HERC5, UBE2L6, AZI2, IFI16, TRIM56, |
| (GO: 0032606) | | IRF7, ISG15, TREX1 |
| Cellular response to | 0.0001 | SP100, OAS1, CCL2, OAS3, OAS2, STAT1, GBP1, |
| interferon-gamma | | TRIM25, NMI, MT2A, TRIM21, OASL, CASP1, |
| (GO: 0071346) | | CDC42EP2, IFNGR2, PARP14, GBP6, IRF7, TRIM34, |
| | | CCL4L1 |
| Regulation of type I | 0.0003 | TLR8, DDX58, DHX58, IFIH1, STAT1, TRIM25, NMI, |
| interferon production | | ZBP1, TRIM21, HERC5, UBE2L6, IFI16, TRIM56, IRF7, |
| (GO: 0032479) | | ISG15, TREX1 |
| Interferon-alpha | 0.0022 | TLR8, DDX58, IFIH1, STAT1, NMI, AZI2, IRF7 |
| production | | |
| (GO: 0032607) | | |

TABLE 4
Differentially expressed genes enriched in immune and inflammatory response

| Description (GO ID) | p-adjust | Genes |
|---|---|---|
| Regulation of innate | 2.6E−06 | LTF, UBE2D1, FCGR2B, NLRC4, SAMHD1, TLR8, |
| immune response | | DDX58, DHX58, MAP2K6, IFIH1, STAT1, TRIM6, NMI, |
| (GO: 0045088) | | ZBP1, RSAD2, RNASEL, DDX60, SERPING1, LY96, |
| | | IFNGR2, IFI16, AIM2, CLEC4E, CLEC4D, FADD, |
| | | STAT2, CLEC7A, PARP14, NLRP6, USP18, IRF7, |
| | | PLSCR1, LILRA2 |
| Negative regulation | 0.0002 | LTF, HMGB3, FCGR2B, SMAD7, CCL2, DHX58, GBP1, |
| of immune system | | NMI, BTN2A2, BST2, ANXA1, GPR55, THBS1, |
| process | | SERPING1, LY96, SAMSN1, HIST1H4H, C1QC, FCRLB, |
| (GO: 0002683) | | IFI16, FADD, PARP14, NLRP6, ZFPM1, PLCB1, CNR2, |
| | | LILRA2, HIST1H4E |
| Positive regulation | 0.0009 | LTF, UBE2D1, NLRC4, TLR8, DDX58, DHX58, |
| of innate immune | | MAP2K6, IFIH1, TRIM6, ZBP1, RSAD2, DDX60, LY96, |
| response | | IFI16, AIM2, CLEC4E, CLEC4D, FADD, CLEC7A, |
| (GO: 0045089) | | NLRP6, IRF7, PLSCR1, LILRA2 |
| Activation of innate | 0.0016 | LTF, UBE2D1, NLRC4, TLR8, DDX58, DHX58, |
| immune response | | MAP2K6, IFIH1, RSAD2, DDX60, LY96, IFI16, AIM2, |
| (GO: 0002218) | | CLEC4E, CLEC4D, FADD, CLEC7A, NLRP6, IRF7, |
| | | LILRA2 |
| Negative regulation | 0.0018 | FCGR2B, SMAD7, DHX58, NMI, BST2, ANXA1, |
| of immune response | | SERPING1, SAMSN1, FCRLB, IFI16, PARP14, NLRP6 |
| (GO: 0050777) | | |
| Regulation of | 0.002 | TFRC, FCGR2B, SMAD7, GPI, DDX58, DHX58, STAT1, |
| immune effector | | NR4A3, TRIM6, IL1B, EIF2AK4, BST2, RSAD2, |
| process | | ANXA1, DDX60, HERC5, SERPING1, C1QC, AIM2, |
| (GO: 0002697) | | FADD, C1QB, C1QA, C4B, APOBEC3G |
| Regulation of | 0.004 | TFRC, FCGR2B, SMAD7, TNFSF13B, IL1B, EIF2AK4, |
| adaptive immune | | RSAD2, ANXA1, SAMSN1, FADD, IRF7 |
| response | | |
| (GO: 0002819) | | |
| Innate immune | 0.0094 | LTF, UBE2D1, TLR8, DDX58, DHX58, MAP2K6, IFIH1, |
| response-activating | | RSAD2, DDX60, LY96, CLEC4E, CLEC4D, FADD, |
| signal transduction | | CLEC7A, NLRP6, IRF7, LILRA2 |
| (GO: 0002758) | | |
| Inflammatory cell | 0.0032 | FASLG, ANXA1, CCR5, IRF7 |
| apoptotic process | | |
| (GO: 0006925) | | |
| Inflammasome | 0.0101 | NLRC4, CASP1, AIM2, NLRP6, CASP4 |
| complex | | |
| (GO: 0061702) | | |

TABLE 5
Differentially expressed genes enriched in apoptosis

| Description (GO ID) | p-adjust | Genes |
|---|---|---|
| Activation of cysteine-type | 0.0002 | CASP10, NLRC4, FASLG, TNFSF10, HIP1R, |
| endopeptidase activity | | CASP1, PMAIP1, SNCA, IFI27, NKX3-1, FADD, |
| involved in apoptotic | | EIF2AK3, TNFSF15, CASP4 |
| process (GO: 0006919) | | |
| Regulation of cysteine-type | 0.0003 | CASP10, PLAUR, PTGS2, NLRC4, MMP9, GPI, |
| endopeptidase activity | | FASLG, TNFSF10, TNFSF14, IFI6, HIP1R, |
| involved in apoptotic | | CASP1, THBS1, PMAIP1, SNCA, IFI27, NKX3-1, |
| process (GO: 0043281) | | FADD, EIF2AK3, TNFSF15, CASP4, CARD16 |
| Regulation of extrinsic | 0.0031 | LTBR, TNFSF10, G0S2, RBCK1, THBS1, |
| apoptotic signaling pathway | | PMAIP1, ATF3, FADD, PTEN |
| via death domain receptors | | |
| (GO: 1902041) | | |
| Positive regulation of | 0.0064 | CASP10, NLRC4, FASLG, TNFSF10, HIP1R, |
| extrinsic apoptotic signaling | | CASP1, PMAIP1, SNCA, IFI27, NKX3-1, FADD, |
| pathway (GO: 2001238) | | EIF2AK3, TNFSF15, CASP4 |
| Positive regulation of | 0.0089 | BRCA1, SP100, FASLG, TNFSF10, THBS1, |
| cysteine-type endopeptidase | | PMAIP1, ATF3, FADD, PTEN |
| activity involved in | | |
| apoptotic process | | |
| (GO: 2001238) | | |
| Regulation of apoptotic | 0.0091 | PLAUR, BRCA1, YBX3, SP100, PTGS2, MMP9, |
| signaling pathway | | SEPT4, LTBR, FASLG, TNFSF10, G0S2, IL1B, |
| (GO: 2001233) | | RBCK1, IFI6, HIP1R, HRK, THBS1, PMAIP1, |
| | | PGAP2, ATF3, NKX3-1, FADD, CX3CR1, PTEN, |
| | | EIF2AK3, PRKN, NANOS3 |
| Regulation of extrinsic | 0.0098 | BRCA1, SP100, LTBR, FASLG, TNFSF10, G0S2, |
| apoptotic signaling pathway | | IL1B, RBCK1, IFI6, THBS1, PMAIP1, ATF3, |
| (GO: 2001236) | | FADD, CX3CR1, PTEN |
| Positive regulation of | 0.0099 | THBS1, PMAIP1, ATF3, FADD, PTEN |
| extrinsic apoptotic signaling | | |
| pathway via death domain | | |
| receptors (GO: 1902043) | | |
| Positive regulation of | 0.0111 | PLAUR, MMP9, SEPT4, LTBR, FASLG, |
| apoptotic signaling pathway | | TNFSF10, G0S2, RBCK1, HIP1R, HRK, THBS1, |
| (GO: 2001235) | | PMAIP1, ATF3, NKX3-1, FADD, PTEN |
| Negative regulation of | 0.0332 | PLAUR, PTGS2, NLRC4, MMP9, GPI, TNFSF14, |
| cysteine-type endopeptidase | | IFI6, THBS1, SNCA, CARD16 |
| activity involved in | | |
| apoptotic process | | |
| (GO: 0043154) | | |

TABLE 6
Differentially expressed genes enriched in myeloid differentiation

| Description (GO ID) | p-adjust | Genes |
|---|---|---|
| Myeloid cell | 1.01E−05 | ITGA2B, LTF, HMGB3, TFRC, OSTM1, MMP9, |
| differentiation | | LTBR, SMAD5, STAT1, EPAS1, NR4A3, KLF2, |
| (GO: 0030099) | | LIF, GPR55, THBS1, MOV10, BATF, IL34, KIT, |
| | | HIST1H4H, C1QC, IFI16, FADD, BATF2, ZFPM1, |
| | | IRF7, SMIM1, EFNA4, HIST1H3E, HIST1H4E |
| Myeloid leukocyte | 0.0001 | LTF, TFRC, OSTM1, MMP9, LTBR, LIF, GPR55, |
| differentiation | | BATF, IL34, KIT, C1QC, IFI16, FADD, BATF2, |
| (GO: 0002573) | | ZFPM1, IRF7, EFNA4 |
| Leukocyte | 0.0001 | LTF, HMGB3, KLF6, TFRC, FCGR2B, OSTM1, |
| differentiation | | MMP9, SMAD7, TNFSF8, LTBR, CD83, SOS1, |
| (GO: 0002521) | | EGR1, FLT3, LIF, RSAD2, ANXA1, GPR55, |
| | | BATF, IL34, KIT, C1QC, AZI2, IFI16, CLEC4E, |
| | | CLEC4D, FADD, BATF2, ZFPM1, IRF7, EFNA4 |
| Dendritic cell | 0.0015 | FCGR2B, LTBR, FLT3, BATF, AZI2, BATF2 |
| differentiation | | |
| regulation of myeloid | | |
| cell differentiation | | |
| (GO: 0097028) | | |
| Regulation of myeloid | 0.0021 | ITGA2B, LTF, HMGB3, STAT1, NR4A3, LIF, |
| cell differentiation | | GPR55, THBS1, MOV10, IL34, HIST1H4H, |
| (GO: 0045637) | | C1QC, FADD, ZFPM1, IRF7, HIST1H3E, |
| | | HIST1H4E |
| Megakaryocyte, | 0.0021 | ITGA2B, NR4A3, THBS1, MOV10, KIT, |
| platelet differentiation | | HIST1H4H, ZFPM1, HIST1H3E, HIST1H4E |
| (GO: 0030219) | | |
| Regulation of | 0.0028 | ITGA2B, NR4A3, THBS1, MOV10, HIST1H4H, |
| megakaryocyte | | ZFPM1, HIST1H3E, HIST1H4E |
| differentiation | | |
| (GO: 0045652) | | |
| Regulation of | 0.0028 | LIF, IL34, C1QC, FADD |
| macrophage | | |
| differentiation | | |
| (GO: 0045649) | | |
| Positive regulation | 0.0032 | LIF, IL34, FADD |
| of macrophage | | |
| differentiation | | |
| (GO: 0045651) | | |
| Negative regulation | 0.0072 | LTF, HMGB3, FCGR2B, SMAD7, ANXA1, |
| of leukocyte | | GPR55, C1QC, ZFPM1 |
| differentiation | | |
| (GO: 1902106) | | |

TABLE 7
Treatment-related adverse events

| | | | |
|---|---|---|---|
| Grade 1 | Grade 2 | Grade 3 | Grade 4 |

Hematological

| | | | | |
|---|---|---|---|---|
| Leukopenia | 0 | 2 (22.2%) | 1 (11.1%) | 0 |
| Anemia | 1 (11.1%) | 2 (22.2%) | 1 (11.1%) | 1 (11.1%) |
| Thrombocytopenia | 1 (11.1%) | 0 | 1 (11.1%) | 0 |

Non-hematological

| | | | | |
|---|---|---|---|---|
| Depression | 1 (11.1%) | 0 | 0 | 0 |
| Alopecia | 3 (33.3%) | 0 | 0 | 0 |
| Bone pain | 1 (11.1%) | 0 | 0 | 0 |
| Neuropathy | 0 | 2 (22.2%) | 0 | 0 |
| Headache | 0 | 1 (11.1%) | 0 | 0 |
| Insomnia | 0 | 1 (11.1%) | 0 | 0 |
| Mucositis | 2 (22.2%) | 0 | 0 | 0 |
| Dizziness | 2 (22.2%) | 0 | 0 | 0 |
| Transaminitis | 0 | 1 (11.1%) | 1 (11.1%) | 0 |

With regards to the dosing, all but one patient (the one with fatty liver) received planned dose escalation during the early phase of treatment. Subsequent dose reduction occurred in four patients during the first year of treatment, two due to excellent response (PBCR), one due to grade 3/4 hematological toxicity, and one due to transient grade 2 transaminitis. As for the dosing schedule, it was shifted to once every month in three of the seven continuously treated patients due to either PBCR (two patients) or the treatment duration exceeding one year (one case).

### Example 3: Treatment of ET patients

Essential Thrombocythemia (ET) is a chronic myeloproliferative neoplasm (MPN) characterized by thrombocytosis. Patients with ET are at higher risk of thrombosis and hemorrhage. They also have disease-related symptoms, which may be difficult to manage. Therapeutic approaches address risks of thrombosis and hemorrhage, without increasing transformation into post-ET myelofibrosis (PET-MF) or acute myeloid leukemia (AML). Low-dose aspirin with hydroxyurea (HU) is recommended as first-line therapy in high-risk patients, supported by data from randomized trials. Approximately 20-40% of ET patients become HU intolerant or resistant, while patients with resistance appear to be at increased risk of disease transformation and reduced overall survival. No prospective clinical trial data exist to guide management of ET patients who are HU resistant or intolerant; treatment options are limited, and several second-line treatment options are associated with increased risk of disease transformation. Established second-line options include interferon alpha (IFN-α) and anagrelide (ANA).

Inclusion criteria for the study includes, among others: (1) subjects diagnosed with high risk ET (either older than 60 years and JAK2V617 positive at screening, or having disease related thrombosis or hemorrhage in the past), diagnosed according to the World Health Organization (WHO) 2016 criteria; (2) Interferon treatment-naïve; (3) Documented resistance/intolerance to prior HU for ET, as defined by ELN criteria; (4) Platelets >450×10⁹/L at screening; (50 WBC >10×10⁹/L at screening; (6) HGB ≥11 g/dL at screening for males and 10 g/dL at screening for females; and (7) Neutrophil count ≥1.0×10⁹/L at screening.

Ropeginterferon-alfa will be administered subcutaneously during the study visits every 2weeks in the clinic. Subjects will receive an initial dose of 250 μg at Week 0, 350 μg at Week 2, and then 500 μg at Week 4, and will remain at 500 μg until at least Week 52. The dose can be further adjusted to prior dose for safety and tolerability reasons, but should preferably remain fixed for the treatment period.

The 2013 ELN and International Working Group-Myeloproliferative Neoplasms Research and Treatment (IWG-MRT) provides 4 response categories for evaluation of response in ET. Complete response requires 1) resolution of disease signs and improvement in symptoms (≥10-point decrease in the MPN-SAF TSS for at least 12 weeks); 2) normalization of peripheral blood counts for at least 12 weeks; 3) absence of vascular events and disease progression; and 4) disappearance of bone marrow histological abnormalities. ELN Response Criteria will be employed to assess response, defined as: (1) peripheral blood count remission (platelets ≤400×10⁹/L and white blood cells [WBCs] <9.5×10⁹/L), (2) improvement or non-progression in disease-related signs splenomegly), (3) large symptoms improvement based on the Myeloproliferative Neoplasm Symptom Assessment Form Total Symptom Score (MPN-SAF TSS), and (4) absence of hemorrhagic or thrombotic events. Large symptom improvement for ET subjects is defined as follows: (1) Baseline TSS scores >20: 10-points reduction in TSS score; (2) Baseline TSS scores 15-19, inclusive: 5-points reduction in TSS score; (3) Baseline TSS scores 10-14, inclusive: TSS score decreases to ≤10; or (4) Baseline TSS score <10: TSS score stays <10.

Other endpoints assessed will include safety and change of MPL, JAK-2, or CALR allelic

burden over time. Evaluation of safety will include assessing vital signs, clinical safety laboratory tests, physical examinations, ECG evaluation, heart ECHO, lung X-ray, ECOG performance status, ocular examination, and adverse events (according to Common Terminology Criteria for Adverse Events).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential

characteristics of the described embodiments, and without departing from the spirit and scope thereof, can make various changes and modifications of the embodiments to adapt it to various usages and conditions. Thus, other embodiments are also within the claims.

What is claimed is:

1. A method of treating essential thrombocythemia in a subject or a subject in need thereof comprising
administering to the subject or the subject in need thereof, a pegylated interferon-α at a regular interval of every 2 to 8 weeks, wherein the subject or the subject in need thereof is administered a first dose including between 250 to 500 μg of the pegylated interferon-α, wherein the pegylated interferon-α includes the following formula: 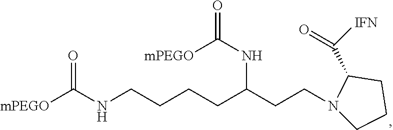
wherein each mPEG includes a molecular weight of 20 kD, and IFN is an interferon-α_{2b}.

2. The method of claim 1, wherein the first dose is between 350 to 500 μg.

3. The method of claim 1, wherein the subject is administered a second dose of the pegylated interferon-α at between 2 to 8 weeks after the first dose without an intervening dose, wherein the second dose includes 50 to 250 μg higher than the first dose, and the second dose being no greater than 500 μg.

4. The method of claim 3, wherein the first dose is 250 μg and the second dose is 350 μg of the pegylated interferon-α.

5. The method of claim 3, wherein the subject is administered a third dose or subsequent doses of the pegylated interferon-a at between 2 to 8 weeks after the second dose without an intervening dose, wherein the third dose or the subsequent doses include between 50 to 200 μg higher than the second dose, and the third dose or the subsequent doses being no greater than 500 μg.

6. The method of claim 5, wherein the first dose is 250 μg, the second dose is 350 μg, and the third dose or the subsequent doses is 500 μg of the pegylated interferon-α.

7. The method of claim 5, wherein the third dose or the subsequent doses include a constant dosage between 250 μg to 500 μg of the pegylated interferon-a.

8. The method of claim 5, wherein the third dose or the subsequent doses are titrated between 250-500 μg per dose of the pegylated interferon-α.

9. The method of claim 1, wherein the pegylated interferon-a has one or more properties comprising:
(i) a median Tₘₐₓ in the range of 3 to 6 days following administration of multiple 50 to 540 μg doses of the conjugate once every two weeks to subjects;
(ii) a mean T_{1/2} in the range of 6 to 10 days following administration of multiple 50 to 540 μg doses of the conjugate once every two weeks to subjects; and
(iii) an individual maximum tolerated dose of at least 500 μg once every 2 to 4 weeks in subjects.

10. The method of claim 1, wherein the subject or the subject in need thereof has:
one or more responses; and/or
one or more disease improvements comprising criteria of European Leukemia Net and/or International Working Group, after administration of the pegylated interferon-α.

11. The method of claim 10, wherein the one or more responses, and/or the one or more disease improvements of European Leukemia Net or International Working Group criteria comprise:
decrease of JAK2V617F allelic burden from baseline;
decrease of CALR and MPL allelic burden from baseline;
normalization of spleen and/or liver, wherein said normalization include lack or no progression of palpable spleen and/or liver;
normal or reduction in spleen and/or liver size;
blood platelet count ≤400×10⁹/L;
white blood cell count <10×10⁹/L;
hematocrit <45%;
lack of or no progression of leukoerythroblastosis;
lack of progression of disease, or improvements of symptoms assessed according to MPN-SAF-TSS score;
lack or reduction of hemorrhagic and/or thrombotic event;
lack or reduction of phlebotomy;
bone marrow histological remission including disappearance of megakaryocyte hyperplasia, and absence of >grade 1 reticulin fibrosis, or presence of age-adjusted normocellularity and disappearance of trilinear hyperplasia, and absence of >grade 1 reticulin fibrosis; and/or molecular remission or response.

12. The method of claim 11, wherein the one or more responses, and/or the one or more disease improvements of European Leukemia Net or International Working Group criteria occurs at a time point comprising on month 3, 6, 9, 12, 18, 24, 30, 36, 42, or 48; or by year 1, 2, 3, 4, 5,or 6, after administration of the first dose of pegylated interferon-α.

13. The method of claim 5, wherein the subject or the subject in need thereof continues to be administered of pegylated interferon-a until the subject or the subject in need thereof exhibits (i) normalization of at least one hematological parameter including hematocrit less than or equal to 45%, white blood cell counts less than or equal to 10×10⁹/L, and/or platelet counts less than or equal to 400×10⁹/L; and
(ii) reduction of JAK2V617F allelic burden of at least 20%, 25%, 30%, 35%, 40%, 45%, 50%, 55%, 60%, 65%, 70%, 75%, 80%, 85%, 90%, 95%, or 99% or more as compared to the JAK2V617F allelic burden as compared to before administration of the pegylated interferon-a.

14. The method of claim 5, wherein the subject or the subject in need thereof continues to be administered of pegylated interferon-a until the subject or the subject in need thereof has one or more disease improvements comprising criteria of European Leukemia Net or International Working Group after administration of the pegylated interferon-α.

15. The method of claim 14, wherein the one or more responses, and/or the one or more disease improvements of European Leukemia Net or International Working Group criteria comprise:
decrease of JAK2V617F allelic burden from baseline;
decrease of CALR and MPL allelic burden from baseline;
normalization of spleen and/or liver, wherein said normalization include lack or no progression of palpable spleen and/or liver;
normal or reduction in spleen and/or liver size;
blood platelet count ≤400×10⁹/L;
white blood cell count <10×10⁹/L;
hematocrit <45%;
lack of or no progression of leukoerythroblastosis;
lack of progression of disease, or improvements of symptoms assessed according to MPN-SAF-TSS score;
lack or reduction of hemorrhagic and/or thrombotic event;
lack or reduction of phlebotomy;
bone marrow histological remission including disappearance of megakaryocyte hyperplasia, and absence of >grade 1 reticulin fibrosis, or presence of age-adjusted normocellularity and disappearance of trilinear hyperplasia, and absence of >grade 1 reticulin fibrosis; and/or molecular remission or response.

16. The method of claim 5, wherein the subject or the subject in need thereof exhibits the following:
peripheral blood count remission including platelets ≤400×10⁹/L and white blood cells (WBC)<9.5×10⁹/L;
improvement or non-progression in splenomegaly;
symptoms improvement or maintain non-progression based on MPN-SAF TSS; and
lack or reduction of hemorrhagic or thrombotic events;
at month 3, 6, 9, or 12 after administration of the first dose of pegylated interferon-a.

17. The method of claim 1, wherein a decrease in one or more of TNFα, TNFβ, IFNγ, IL4, and/or IL12 levels is detected in the subject or the subject in need thereof, after administration of the first dose of the pegylated interferon-α. 18. The method of claim 1, wherein an increase in hepcidin level is detected in the subject or the subject in need thereof, after administration of the first dose of the pegylated interferon-α.
